(19) United States
(12) Patent Application Publication
(10) Pub. No.: US 2025/0000513 A1
(43) Pub. Date: Jan. 2, 2025

(54) MEDICAL OCCLUSION DEVICE AND SYSTEM COMPRISING THE MEDICAL OCCLUSION DEVICE AND A CATHETER

(71) Applicant: BIOTRONIK AG, Buelach (CH)

(72) Inventor: Jorge Calisse, Berlin (DE)

(21) Appl. No.: 18/709,762

(22) PCT Filed: Nov. 30, 2022
(86) PCT No.: PCT/EP2022/083757

§ 371 Date: May 13, 2024

(30) Foreign Application Priority Data
Nov. 30, 2021 (EP) 21211222.1

(51) Int. Cl. A61B 17/12

(52) U.S. Cl. CPC A61B 17/12122; A61B 17/12177

(57) ABSTRACT

A preferred medical occlusion device has a compressed framework state and is configured to expand in an expanded framework. A cover covers at least a proximal framework section. A first connector part is connected to a distal framework section. A second connector part is configured to be connectable with the first connector part. A protrusion includes at least start middle and end protrusion sections. The end protrusion section projects radially beyond the framework in its expanded framework state. The start protrusion section is attached to the second connector part and the proximal framework section includes a coupling element configured to provide a releasable connection with a catheter.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2022/083757, which was filed Nov. 30, 2022, which application claimed priority from EP application Ser. No. 21/211,222.1, which was filed Nov. 30, 2021.

### FIELD OF THE INVENTION

The invention is generally directed to a medical occlusion device for occluding a body lumen or a cardiovascular structure, for example a left atrial appendage (LAA), and to a system including the medical occlusion device and a catheter.

### BACKGROUND

Medical occlusion devices are useful for occluding cardiovascular structures or defects in which an end of the medical occlusion device is in the path of blood flow, such as closure of the LAA (left atrial appendage), Atrial and Ventricular Septal Defects (ASD, VSD), and Patent Ductus Arteriosus (PDA) and the like. Left atrial appendage occlusion (LAAO) or left atrial appendage closure (LAAC) can reduce the risk of left atrial appendage blood clots (thrombi) from entering the bloodstream and causing a stroke in patients with non-valvular atrial fibrillation.

A wide variety of intravascular devices are used in treating specific conditions. In certain circumstances, it may be necessary to occlude an abnormal opening in a patient's vessel, such as an abnormal opening between chambers of the heart, a channel, a hole, a cavity, or the like, so as to stop blood flow therethrough. For example, atrial fibrillation may result in the formation of a blood clot in LAA, which may become dislodged and enter the blood stream. By occluding the LAA, the release of blood clots from the LAA may be significantly reduced, if not eliminated. Various techniques have been developed to occlude the LAA. For example, balloon-like devices have been developed that are configured to be implanted completely within the cavity of the LAA, while surgical techniques have also been developed where the cavity of the LAA is inverted and surgically closed.

Document U.S. Pat. No. 10,660,647 B2 describes a device for closing the LAA. The device includes a self-expandable stent framework, which can be expanded automatically from a compressed state into an expanded state, and a flexible material layer fixed to the stent framework, wherein the material layer is substantially stretched in the expanded state of the stent framework. Further, the device has a flexible and elongate pulling element fixed to a flexible material layer fixedly connected to a self-expanding stent. The closure implant is configured to be drawn back into the lumen of a catheter by the pulling element, and in so doing the stent framework is transferred into a compressed state. The device enables a monitored and controllable release of the closure implant.

Devices for closing the LAA often need to be repositioned many times before device release. This recapture and repositioning procedure is time consuming and sometimes a new device is needed because of permanent device deformations that occur during recapturing it into the delivery system and releasing it again.

Further, document EP 2 779 910 B1 discloses a medical occlusion device for LAA closure having a tubular structure including a plurality of braided strands forming a tubular structure with a first disc-shaped expanded volume portion, a second cylindrically shaped expanded volume portion displaced from the first expanded volume portion and connected to the first expanded volume portion by a connection portion. The first expanded volume portion of the device has a diameter that is intended to abut the adjacent wall surrounding the LAA to prevent device movement toward the second expanded volume portion and to assist in sealing the aperture. This means that the first expanded volume portion is oversized so as to be capable of overlying the ostium or opening of the LAA and lying adjacent to, and in flush contact with, the wall of the atrium. The diameter of the second expanded volume portion is less than the diameter of the first volume portion so as to fit in the LAA. The first expanded volume portion is flexible so as to be capable of conforming to the curvature of the wall of the atrium in LAA applications or other vascular structures in other applications. It is disclosed in this document that one or both expanded volume portions may be flat disks or disks having a convex distal section, or the device may include a smaller diameter central cylindrical portion between two larger diameter disks. The above device has a complex design having a quite large diameter in the compressed state being disadvantageous for implantation procedure.

The drawbacks of LAA closure device positioning failures are correlated with serious adverse events, for example strokes due to formation and release of thrombi—not solving the current patient problem-, losing device anchoring with the consequences of plugging the mitral valve and patient death or leakage between device and LAA based on wrong position. Accordingly, a key factor for successful patient treatment in connection with closure of LAA is the correct and permanently reliable positioning of the device.

Hence, a good device alignment at the LAA ostium is mandatory to avoid serious adverse events. According to FIG. 1 state of the art systems 1 with a closure device 1c are firmly connected by a connecting element 1b to the catheter 1a during implantation. With this system the best position is very difficult to achieve and depends on many factors such as position of septal puncture, anatomic position and curvature of LAA, preselection of access system, device type, etc. The current control of the position is done visually with fluoroscopy and/or TEE (transesophageal echocardiography). The most important criterion for device release before disconnecting the device from the delivery system is to control the position of the LAA closure device at the ostium.

### SUMMARY OF THE INVENTION

Preferred embodiments provide an improved LAA closure device having improved positioning properties as the most important device release criterion before disconnecting the device from delivery system is to control the position of LAA closure device at the ostium. A preferred medical occlusion device for occluding a body lumen or a cardiovascular structure includes a framework with a longitudinal framework axis (A). The framework has a compressed framework state and is configured to expand in an expanded framework state when the medical occlusion device is not being compressed. The framework includes a distal framework section, a proximal framework section and an intermediate framework section between the proximal framework section and the distal framework section. A cover covers at least the proximal framework section. A first connector part is connected to the framework and is located in the distal framework section. A second connector part is configured to be connectable with the first connector part. At least one protrusion is configured to position and/or anchor the medical occlusion device at an ostium of the body lumen or the cardiovascular structure. The protrusion includes at least one start protrusion section, at least one middle protrusion section and at least one end protrusion section. At least one of the at least one end protrusion sections projects radially beyond the framework in its expanded framework state with respect to the longitudinal framework axis (A). The start protrusion section is attached to the second connector part and the proximal framework section includes a coupling element configured to provide a releasable connection with a catheter.

### BRIEF DESCRIPTION OF THE DRAWINGS

FIG. 1 shows a medical occlusion device according to prior art in the expanded state located within an LAA, a cross section of a patient's heart and a catheter,

FIG. 2 depicts a first embodiment of a medical occlusion device in a side view in the expanded state located within an LAA shown in a cross section during implantation (with distal section of catheter),

FIG. 3 shows an embodiment of the medical occlusion device in a top view in the expanded state,

FIG. 4 depicts the embodiment of the medical occlusion device of FIG. 2 in a perspective side view in the expanded state,

FIG. 5 depicts the embodiment of the medical occlusion device of FIG. 2 in a cross section in the compressed state within a sleeve of the catheter,

FIG. 6 depicts a second embodiment of the medical occlusion device in a perspective side view in the expanded state,

FIG. 7 depicts the embodiment of the medical occlusion device of FIG. 6 in another perspective side view in the expanded state attached to a catheter,

FIG. 8 depicts the embodiment of the medical occlusion device of FIG. 6 in a side view in the expanded state located within an LAA shown in a cross section during implantation (with distal section of catheter),

FIG. 9 depicts the embodiment of the medical occlusion device of FIG. 6 in an exploded side view in the expanded state,

FIG. 10 depicts a third embodiment of the medical occlusion device in a perspective side view in the expanded state,

FIG. 11 depicts the embodiment of the medical occlusion device of FIG. 10 in a perspective side view in the expanded state,

FIG. 12 depicts the embodiment of the medical occlusion device of FIG. 10 in a side view in the expanded state located within an LAA shown in a cross section during implantation (with distal section of catheter),

FIG. 13 depicts the embodiment of the medical occlusion device of FIG. 10 in a side view in the expanded state,

FIG. 14 depicts the embodiment of the medical occlusion device of FIG. 10 in a cross section in the compressed state within a sleeve of the catheter,

FIG. 15 depicts a fourth embodiment of the medical occlusion device in a cross-sectional view in the expanded state,

FIG. 16 shows the embodiment of FIG. 15 in a side view in the expanded state.

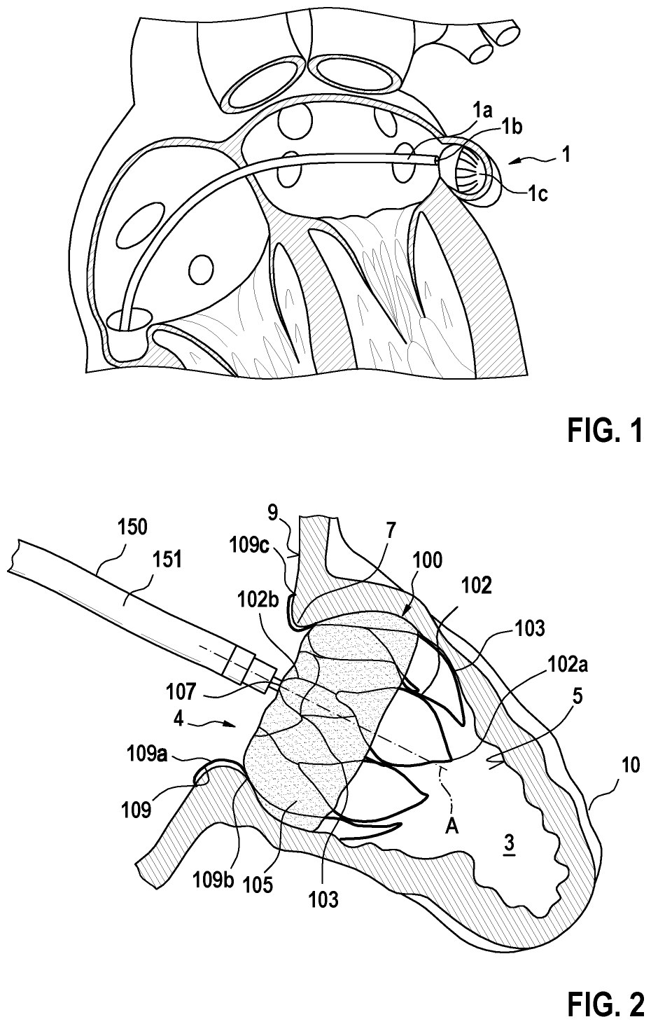

### DETAILED DESCRIPTION OF THE PREFERRED EMBODIMENTS

A medical occlusion device suitable for occluding (an abnormality or defect in) a body lumen or a cardiovascular structure, for example for occluding a left atrial appendage, at least includes
a framework having a longitudinal framework axis wherein the framework has a compressed framework state and is configured to expand in an expanded framework state when the medical occlusion device is not being compressed (unconstrained),
a cover covering at least a part of the framework section, and
at least one protrusion for positioning and/or anchoring the medical occlusion device at (an ostium of) the body lumen or the cardiovascular structure, for example at an ostium of the left atrial appendage.

The framework has a distal framework section, a proximal framework section and an intermediate framework section between the proximal framework section and the distal framework section.

The framework can be built from a plurality of struts (and spaces between the struts). The framework has an open or closed framework structure. Open framework structure means a framework having an opening at the distal framework section. Thus, an open framework being built from a plurality of struts has spaces between the struts and an opening at the distal framework section. The framework can be also named an open or closed cage, wherein the cage being formed by the plurality of struts. The plurality of struts can form a cell structure having a plurality of cells. The plurality of struts can extend from the proximal section to the distal section of the framework.

The framework has a longitudinal framework axis (A). The framework has a vertical framework axis and a lateral framework axis, wherein the vertical framework axis and the lateral framework axis are perpendicular to the longitudinal framework axis.

The proximal framework section is the section of the framework from which a catheter can be connected. The proximal framework section (with its plurality of struts and spaces between the struts) has a crossing point with the longitudinal framework axis. The proximal framework section has an inner and outer side.

The intermediate section (with its plurality of struts and spaces between the struts) has no crossing point with the longitudinal framework axis. The intermediate framework section can be the section of the framework having the outmost expansion in the expanded framework state with respect to the longitudinal axis. The intermediate framework section has an inner and outer side.

The distal framework section (with its plurality of struts and spaces between the struts) either has a crossing point with the longitudinal framework axis if the framework has a closed framework structure. The distal framework section has no crossing point with the longitudinal framework axis if the framework has an open framework structure. In case the framework has an open framework structure the longitudinal framework axis lies within the opening of the open framework structure. The distal framework section can be situated on the opposite side to the proximal framework section. The intermediate framework section has an inner and outer side.

The framework (with its plurality of struts and spaces between the struts) defines an inner framework volume. The framework has an inner framework side and an outer framework side. The inner framework side is adjacent to or in contact with the inner framework volume. The outer framework side can be in contact with an inner surface of a catheter lumen in a delivery state. In the delivery state the framework has a compressed framework state.

The outer framework side can be in contact with the inner surface of the body lumen in an implantation state. In the implantation state the framework has an expanded framework state.

The proximal framework section has an inner proximal framework side and an outer proximal framework side, the intermediate framework section has an inner intermediate framework side and an outer intermediate framework side, and the distal framework section has an inner distal framework side and an outer distal framework side.

The framework has a dimension in a longitudinal direction, wherein the longitudinal direction is the direction along the longitudinal axis. As circumferential direction a direction around the longitudinal framework axis is understood.

The framework can be a self-expandable or self-expanding framework. This is advantageous as a self-expanding framework can more flexibly adapt to the inner surface of the body lumen or the cardiovascular structure, and thus occluding it more efficiently. The (self-expandable) framework can have a plurality of struts extending in the compressed state of the framework along the longitudinal framework axis.

The (self-expandable) framework in its expanded framework state is configured to occlude (the abnormality or defect in) the body lumen or the cardiovascular structure, for example the LAA. The framework with its inner framework volume fills in the expanded framework state the recess or cavity provided by (the abnormality or defect in) the body lumen or the cardiovascular structure, for example the LAA. Accordingly, the self-expandable framework may be flexible to a certain extend in order to adapt to the specific form of (the abnormality or defect in) the body lumen or to the cardiovascular structure, for example to the LAA. After implantation at least parts of the struts are in contact with an inner surface of the (abnormality or defect in the) body lumen or the cardiovascular structure, for example the LAA.

The outer framework side or the cover can be in contact with the inner surface of the abnormality or defect in the body lumen or the cardiovascular structure, for example the LAA. The struts may be directly in contact with the inner surface of (the abnormality or defect in) the body lumen or the cardiovascular structure. The struts may be in contact via the cover covering at least part of the struts. Thereby, at least part of the struts may exert a radial biasing force to the inner surface of the vascular structure.

The cover covers at least a part of the framework section or the whole framework (including the proximal framework section, the intermediate framework section and the distal framework section), preferably the cover covers at least the proximal framework section or the proximal framework section and the intermediate framework section. The cover can cover the inner framework side and/or outer framework side, preferably the outer framework side. The cover can cover at least the outer side of the framework section. The cover can cover at least an outer side of the plurality of struts, preferably in the proximal framework section. The cover covering the outer side enables a more leak-proof closure of the body lumen or the cardiovascular structure, for example the LAA.

The medical occlusion device has at least one, two or three protrusions for positioning and/or anchoring the medical occlusion device at (an ostium of) the body lumen or the cardiovascular structure, for example at (an ostium of) the left atrial appendage. Three protrusions could offer an improved stability compare to only one (circumferential) protrusion. The protrusions are preferably distinct protrusions (not being directly attached to each other).

According to the invention the at least one protrusion is at least adjacent to the proximal framework section. The start protrusion section can be directly attached to at least one of the plurality of struts.

A protrusion has at least one end protrusion sections, at least one middle protrusion sections and at least one start protrusion sections. The middle protrusion section is the part of the protrusion between the start protrusion section and the end protrusion section. The start protrusion section is the part of the protrusion which is attached to the framework or protrudes from the framework. Preferably, the start protrusion section is the part of the protrusion which is attached to at least one strut of the plurality of struts of the framework or protrudes from at least one strut of the plurality of struts of the framework. The middle protrusion section is the section of the protrusion including an inflection point with respect to the start section and the end section. The middle protrusion section can be either partially covered or not covered by the cover. The end protrusion section is the section of the protrusion which enables the positioning and/or anchoring of the medical occlusion device at (an ostium of) the body lumen or the cardiovascular structure, for example (at an ostium of) the left atrial appendage).

The start protrusion sections of one protrusion or several protrusions can be attached to different framework sections or to the same framework section at different heights with respect to the longitudinal framework axis. For example, the start protrusion sections of one protrusion or several protrusions can be attached to the framework or to the plurality of struts at different distances from the distal framework section or the proximal framework section.

This enables a space-saving assembly in the compressed framework state.

According to the invention, the at least one protrusion projects radially beyond the framework in its expanded state. Projecting radially beyond the framework in its expanded state especially means that the middle protrusion section of the at least one protrusion is (approximately) at the same level as the proximal framework section with regard to the longitudinal framework axis and the end protrusion section projects beyond the middle protrusion section of the framework with respect to the longitudinal framework axis in the expanded state. This means the protrusion can have a dimension in longitudinal direction which is greater than the dimension of the framework in longitudinal direction in the expanded state and/or compressed state. This needs to be considered during implantation of the medical occlusion device.

The at least one protrusion can be at least partially covered by the cover or cannot be covered by the cover at all. The end protrusion section can be at least partially not covered by the cover or the end protrusion section cannot be covered by the cover. However, the end protrusion section could be covered by another covering layer or a coating (e.g. drug based coating or anti-inflammatory coating). At least a part of the middle protrusion section and an end protrusion section can be covered by the cover. The start protrusion section and/or the middle protrusion section can be either covered or not covered by the cover. The at least one protrusion, preferably the end protrusion section, can at least partially protrude through the cover.

In the following the invention is explained with regard to an LAA. The inventive medical occlusion device and system may analogously be used with regard to other body lumens or cardiovascular structures like Atrial and Ventricular Septal Defects (ASD, VSD), Patent Ductus Arteriosus (PDA) and the like. They are acting in the same way concerning closure and positioning.

The at least one protrusion of the medical occlusion device is anchored at the rim of the ostium. Thereby, the longitudinal framework axis of the medical occlusion device is aligned with the LAA or with the ostium. This provides a visual control and aids fixation. At least two protrusions prevent a full movement of the medical occlusion device into the LAA. At least one protrusion fixes the medical occlusion device with its proximal end at the ostium of the LAA, wherein the distal framework end of the medical occlusion device protrudes into the LAA so that the LAA is occluded. The advantage of the at least one protrusion is further, that it only needs limited material and, accordingly, increases the cross section of the medical occlusion device in the compressed state only marginally. The number of protrusions depends on the shape of the protrusions. Three protrusions are favorable to get a good stability and a good apposition at difficult LAA shapes. If more protrusions are used, they may be very flexible in order to achieve a good apposition of all protrusions at the ostium. Among other things, the protrusion prevents the implant from sliding too deeply into the LAA. This is advantageous over protrusions that just prevent the implant from sliding out of the LAA.

In one embodiment, the protrusion can be a loop. The number of loops can be between 2 and 9, in another embodiment between 2 and 4. In one embodiment the at least one loop projects radially beyond the framework in its expanded state such that a middle section of the at least one loop is approximately at the same level as the proximal framework section with regard to the longitudinal framework axis or projects beyond the proximal framework section with regard to the longitudinal framework axis in the expanded state. This middle section extends approximately around half of the entire length of each loop. The middle section may extend along the most proximal third of the framework with regard to its longitudinal framework axis or proximal from the proximal section. Accordingly, at least two third of the framework may protrude into the LAA thereby very reliably occluding the LAA.

In one embodiment the middle section of each loop generally extends in circumferential direction around the longitudinal axis. The sections other than the middle section of each loop may generally extend generally in longitudinal direction.

There are two types of loops. A first type of loop is formed such that a first part and a second part are directly attached to the distal section of the framework or the first part and the second part are directly attached to the proximal framework section. This means that the first part and the second part of the same loop are attached to the same section of the framework. A second type of loop is formed such that the first part is directly attached to a first strut of the plurality of struts and the second part is directly attached to a first or second strut of the plurality of struts. In particular, the position of the attachment area of the first part and the attachment area of second part of one loop is spaced from the distal framework section and the proximal framework section. The second type of loop may be attached to the struts of the framework that the first part and the second part of the same loop are located approximately parallel to the same section of the longitudinal framework axis of the framework. The first part and the second part are located equidistantly from the distal framework section or the proximal framework section, wherein the distance from the distal framework section and the 4840*1564911'S proximal framework section may be different. The first part and the second part can be located equidistantly from the distal framework section and the proximal framework section. In another embodiment the first part and the second part of the loop can have a different distance from the distal framework section (or the proximal framework section). In one embodiment the medical occlusion device includes only one type of loops. The second type of loop may be formed such that the second strut is directly adjacent or next but one to the first strut in circumferential direction. The loop of the first and second type may be welded or glued to the framework or the struts or may be integrally formed with struts.

The loop can be designed in such a way that in an expanded state it spans a widened surface compared to the compressed state. The loop can rest on the ostium. The loop can be covered by an (elastic) cover.

In one embodiment, the at least one loop may include radiopaque material for a better fluoroscopy. For example, one section of each loop is made of a radiopaque material in order to visualize the position of the medical occlusion device within the patient's body and to assess the progress of the implantation.

In the framework of the present invention, the notion distal refers to a portion or component of the medical occlusion device that is—with respect to the longitudinal framework axis of the framework, the medical occlusion device and a longitudinal framework axis of a catheter for implantation of the medical occlusion device-more remote from the catheter handle or from the physician that operates the catheter device than a corresponding proximal portion or component that is closer to the handle or physician.

One of the proximal framework section and the distal framework section may include a coupling element for releasable connection with a catheter. Preferably, the proximal framework section includes a coupling element for releasable connection with a catheter.

The cover can cover at least one third of the framework or at least half of the framework. The cover may cover the full framework in the expanded state. Hence, the cover can be understood as a cover for covering the framework at least partially. The cover can cover the framework at its inner or outer side in the expanded state. In particular, the cover can cover the proximal framework section at its outer side (in order to occlude the LAA and to avoid passing of thrombi into the vasculature). The cover may include or may consist of a synthetic material e.g. a polymer such as polyethylene terephthalate (PET), polyamide (PA), Polytetrafluoroethylene (PTFE e.g. Teflon®) and/or polypropylene (PP). These materials can be connected as individual fibres or fibre bundles to form a fabric. Such a fabric can made of the polyester polyethylene terephthalate fibres, which are sold for example under the trade name Dacron®. In an alternative embodiment the cover may include a natural material, such as a (processed) biological tissue, like human and/or animal tissue such as pericardial tissue or collagen or bacterial (nano) cellulose. Preferably, the cover is made from pericardial tissue. Thus, the cover includes or consists of pericardial tissue, preferably decellularized pericardial tissue.

In one embodiment, the medical occlusion device is configured such that the framework and the at least one protrusion form two separate device parts, that the two separate device parts are implanted in two consecutive or subsequent steps and are attached to each other after implantation, for example when the protrusion is already located at the correct position at the ostium of the LAA. This may ease the correct placement of the loop at the ostium (without the voluminous framework).

Therefore, a medical occlusion device for occluding a body lumen or a cardiovascular structure, for example for occluding a left atrial appendage, is described, including a first device part including
a framework having a longitudinal framework axis wherein the framework has a compressed framework state and is configured to expand in an expanded framework state when the medical occlusion device is not being compressed, and wherein the framework has a distal framework section, a proximal framework section and an intermediate framework section between the proximal framework section and the distal framework section,
a cover covering at least the proximal framework section (at least in the expanded framework state), and
a first connector part, and second device part including:
a second connector part, being configured to be connectable with the first connector part of the first device part,
at least one protrusion for positioning and/or anchoring the medical occlusion device at an ostium of the body lumen or the cardiovascular structure, for example at an ostium of the left atrial appendage, having at least one start protrusion section, at least one middle protrusion section and at least one end protrusion section, and wherein at least one of the end protrusion sections projects radially beyond the framework in its expanded framework state with respect to the longitudinal axis, and wherein the start protrusion section of the at least one protrusion is attached to the second connector part.

A method for implanting such a medical occlusion device is descried as well, wherein the second device part is positioned and/or anchored at an ostium of a body lumen or a cardiovascular structure, for example at an ostium of a left atrial appendage, in a first implantation step and in a subsequent second implantation step connecting the first connector of the first device part to the second connector part of the second device part.

In a subsequent second implantation step means either within one operation (e.g. within one operation day) or in another operation.

The first device part can be housed in a first catheter and the second device part can be housed in a second catheter or the first device part and the second device part could be housed in the same catheter.

The method may further include a subsequent third implantation step wherein the first device part is moved out (e.g. pushed out) of the catheter and released from the catheter, thereby expanding the framework of first device part into the expanded framework state.

In one embodiment, at least one loop extends along the dimension of the framework in the longitudinal direction and back in the expanded state. Thereby the loop receives a spring-like property. This supports long-lasting fixation of the loop within the ostium of the LAA.

In one embodiment, the medical occlusion device includes a coupling element for coupling to a catheter during implantation, wherein the coupling element is located at the distal section or the proximal framework section, for example where the first part and the second part of the at least one loop are directly attached to. This is advantageous because the coupling element has a suitable dimension for reliable attachment of the loop, for example by welding or gluing.

In one embodiment, the at least one loop has one section extending within the volume encased by the framework and a second section extending outside the framework in the expanded state. In this state the loop takes up less space than if it fully extends outside the framework.

In one embodiment, the protrusion is a loop. The loop is of the first type and the medical occlusion device includes at least two or three of such loops. In the expanded state the first part and the second part of each loop is attached to the distal section of the framework. Further, each loop (fully) extends outside the framework. From the attachment section of each loop the loop extends in distal longitudinal direction, is then bent upwards and runs parallel to the longitudinal framework axis of the framework. At a position parallel to the proximal framework section, a middle section of the loop is bent by approximately 90° to 120° to the outside thereby forming a protrusion, where the middle section runs generally over approximately 50° to 120° along the circumferential direction with regard to the longitudinal axis. With regard to this embodiment the loop and the framework may form two separate assemblies which may be implanted in two separate steps and attached after implantation.

In another embodiment, the loop is of the first type and the medical occlusion device includes at least two or three loops. The loop has a start loop section, a middle loop section and an end loop section. The middle loop section is the section of the loop including an inflection point with respect to the start loop section and the end loop section. The start loop section is the attachment section of the loop on the framework. In the expanded state the first part and the second part of each loop is attached to the proximal framework section. Further, each loop protrudes (radially) outside the framework. Looking from the start loop section of the loop the middle loop and end loop section protrude in radial direction and the middle section is bent (downwards so that the end loop section runs perpendicular to the longitudinal framework axis of the framework). The middle section of the loop is bent by approximately 90° to 120° to the outside. The middle section runs generally over approximately 20° to 90° along the circumferential direction with regard to the longitudinal axis.

In another embodiment, the loop is of the second type and the medical occlusion device includes at least two or three of such loops. The loop has two start loop sections, namely a first start loop section and a second start loop section, a middle loop section and an end loop section. The middle loop section is the section of the loop including an inflection point with respect to the start loop sections and the end loop section. In the expanded state the first start loop section and the second start loop section of each loop is attached to adjacent struts of the framework, preferably located at the proximal third of the framework. Further, each loop extends partly inside and partly outside the framework. Looking from start loop sections of each loop the start loop section loop extends in proximal longitudinal direction or slightly inclined to it. The middle loop section of the loop is bent by approximately 70° to 120° to the outside. The middle section runs generally over approximately 10° to 70° along the circumferential direction with regard to the longitudinal axis.

In one embodiment, the at least one loop includes at least one material of the group including Nickel-Titanium alloy and any other shape-memory material, absorbable or permanent polymer material, Cobalt-Chromium alloy and stainless steel. In one embodiment, the at least one loop is self-expendable.

In one embodiment, struts of the framework are at least partially made of a shape-memory material. The framework can be produced by laser cutting from a tube or of shape-memory material wire.

Further a system including the above described medical occlusion device and a catheter with a lumen configured to receive the medical occlusion device in the compressed state of the framework. The medical occlusion device is configured to be releasable from the lumen of the catheter by being pushed out or by retracting an outer sleeve, and in so doing the framework is transferred into the expanded state. During sliding out or retraction, first, the loop may expand in order to be aligned with the ostium of the LAA. Then, the distal section of the framework is released from the catheter thereby enabling abutment of the framework to the inner surface of the LAA.

The framework may include a coupling element for releasable attachment of the framework to an inner tube of the catheter. In one embodiment, the medical occlusion device and the catheter are detachably connected by screwing, releasing anchors, clamping or a bayonet lock. Alternatively, the coupling element may include a predetermined breaking point or zone which may be broken once the medical occlusion device is correctly positioned at the ostium of the LAA in order to separate the medical occlusion device from the catheter.

In one embodiment, the medical occlusion device has a pulling element fixed to the framework, wherein the pulling element is configured and fixed to the framework such that the medical occlusion device is drawable back into the lumen of the catheter, and in so doing the framework is transferable into the compressed state.

In one embodiment, the middle section of the at least one loop is angled by an angle between 70° and 110° with regard to both directly adjacent sections in the expanded state. The angled form of the loop allows adaption of the loop to the form of the ostium's rim thereby improving anchoring of the medical occlusion device at the ostium. The angled section may form an undercut. The bent middle section may run along the circumferential direction with regard to the longitudinal framework axis over an angle between 10° and 150°.

In one embodiment, each of the at least one loop two times penetrates the cover. The attachment section of the at least one loop may be covered by the cover in this case. In one embodiment the cover includes at least one or two springs wherein each spring is sized and configured to close-off a through-hole of the cover were the at least one loop penetrates the cover. The spring may have a clover-like form and may integrated within the cover. Further, the spring may encircle the through-hole fully or over an angle of at least 250°.

The following alternative examples should describe the invention in more detail.

A medical occlusion device for occluding a body lumen or a cardiovascular structure, for example for occluding a left atrial appendage, including:
a first device part includes
a framework having a longitudinal framework axis wherein the framework has a compressed framework state and is configured to expand in an expanded framework state when the medical occlusion device is not being compressed, and wherein the framework has a distal framework section, a proximal framework section and an intermediate framework section between the proximal framework section and the distal framework section,
a cover covering at least the proximal framework section, and
a first connector part being connected to the framework; and
a second device part including
a second connector part, being configured to be connectable with the first connector part of the first device part,
at least one protrusion for positioning and/or anchoring the medical occlusion device at an ostium of the body lumen or the cardiovascular structure, for example at an ostium of the left atrial appendage, having at least one start protrusion section, at least one middle protrusion section and at least one end protrusion section, and wherein at least one of the end protrusion sections projects radially beyond the framework in its expanded framework state with respect to the longitudinal framework axis, and wherein the start protrusion section of the at least one protrusion is attached to the second connector part.

The first connector part and/or the second connector part can be located along the longitudinal framework axis.

3The first connector part can be located in the distal framework section.

The framework can have a plurality of struts, preferably extending from the proximal framework section to the distal framework section or vice versa.

The medical occlusion device can have at least 1, 2 or 3 protrusions, preferably 2 to 24 protrusion, more preferably 3 protrusions.

\The end protrusion section can be angled by an angle between 70° and 110°, preferably between 80° and 100°, more preferably between 85° and 90°, most preferably by an angle of 90°, with regard to the middle protrusion section and/or the start protrusion section in the expanded framework state.

A dimension of the at least one protrusion in a longitudinal direction can be greater than the dimension of the framework in the longitudinal direction in the expanded framework state and/or in the compressed framework state.

8The cover preferably includes or consists of a material impermeable for blood cloths, and preferably includes or consists of a polymer or a human and/or animal tissue.

The end protrusion section is preferably blunt or has rounded corner(s) and/or edge(s) and/or tip(s).

The at least one protrusion is preferably a loop, preferably an open or closed loop.

The framework is preferably a self-expandable or self-expanding framework.

The medical occlusion device includes a coupling element for coupling to a catheter, wherein the coupling element is attached to at least one of the plurality of struts.

The coupling element is preferably situated at the distal framework section or the proximal framework section, preferably at the proximal framework section.

The plurality of struts preferably forms a cells structure having a plurality of cells or a net structure having a plurality of meshes.

The cells of the cell structure preferably being diamond-shaped cells.

The plurality of struts can be in the form of a wire or strip.

The plurality of struts can extend from the proximal framework section to the distal framework section or vice versa.

The plurality of struts preferably extends along a longitudinal framework axis in the compressed framework state.

The plurality of struts is preferably at least partially made of a shape-memory material.

The plurality of struts preferably consists of or include a Nickel-Titanium alloy, a Cobalt-Chromium alloy, a polymer or a stainless steel.

The framework or the plurality of struts are preferably produced by laser cutting from a shape-memory material.

The framework preferably has a closed framework structure wherein the distal framework section has a crossing point with the longitudinal framework axis or wherein the framework has an open framework structure wherein the distal framework section has no crossing point with the longitudinal framework axis.

The framework preferably encases an inner framework volume in the form of a sphere or a flattened sphere, preferably an ellipsoid.

The framework preferably is a self-expandable or self-expanding framework.

2The at least one protrusions preferably has at least one, preferably one or two, end protrusion sections and at least one, preferably one or two, middle protrusion sections and at least one, preferably one or two, start protrusion sections.

The middle protrusion section preferably has an inflection point with respect to the start protrusion section and the end protrusion section.

The protrusions are preferably distinct protrusions.

The protrusions preferably are not directly attached to each other.

The at least one protrusion may include or consist of a radiopaque material or at least one radio marker is attached thereto.

The at least one protrusion is preferably not a circumferential protrusion with respect to the longitudinal framework axis.

The end protrusion section, the middle protrusion section and/or the start protrusion section is blunt.

The at least one protrusion can be a wire, strap or a strip.

The at least one protrusion includes a winding.

The at least one protrusion is preferably a loop, preferably a closed or open loop.

The at least one protrusion can have an airfoil-like shape, wing-like shape or lasso-like shape in its expanded state or looks like the cross section through a mushroom or an umbrella.

The end protrusion section is preferably u-shaped, n-shaped, c-shaped, elliptical shaped or circular shaped.

The end protrusion sections are preferably evenly distributed in circumferential direction.

The start protrusion sections of one protrusion or several protrusions can be attached to different framework sections or to the same framework section at different heights with respect to the longitudinal framework axis.

The middle protrusion section of the at least one protrusion is at the same level as the proximal framework section with respect to the longitudinal framework axis.

The at least one protrusion is preferably self-expendable or self-expanding.

The at least one protrusion preferably extends along the dimension of the framework and back in the longitudinal direction in the expanded state.

The at least one start protrusion section is preferably attached to the inner side of the framework or the inner side of the struts.

The start protrusion section is preferably directly attached to the outer side of the struts.

The at least one protrusion preferably has a section extending within the inner framework volume and another section extending outside the framework in the expanded state.

The shape in cross section of the at least one protrusion is preferably circular, elliptic or polygonal with rounded corners.

The start protrusion section is preferably partially covered by the cover.

The at least one protrusion preferably consists of or includes a shape-memory material.

The at least one protrusion preferably consists of or includes a Nickel-Titanium alloy, a Cobalt-Chromium alloy, a polymer or a stainless steel.

The at least one protrusion and the framework or at least one of the plurality of struts of the framework are preferably fixed together by welding, screwing or riveting both together.

The cover is preferably made of flexible material.

The cover preferably includes or consists of a material impermeable for blood clots or thrombi but permeable for blood.

The cover preferably includes or consists of a polymer or a biological tissue, preferably a human and/or animal tissue.

The cover preferably includes or consists of a pericardial tissue, preferably decellularized pericardial tissue.

The end protrusion section is preferably not covered by the cover.

Every protrusion preferably two times penetrates the cover.

The cover preferably covers at least one third of the framework at its proximal framework section or the full framework in the expanded state.

The cover preferably covers the outer side of the struts and spaces between the struts in the expanded framework state.

The cover preferably includes at least one spring being sized and configured to close-off a through-hole of the cover where the at least one protrusion or the coupling element penetrates the cover.

The cover preferably includes at least one spring being sized and configured to close-off a through-hole of the cover where the at least one protrusion protrudes through the cover.

The cover is preferably glued or sutured to the plurality of struts.

The plurality of struts is preferably embedded in the cover.

The medical occlusion device preferably includes a coupling element for coupling to a catheter.

The coupling element is preferably attached to the distal framework section or the proximal framework section.

The coupling element is preferably attached to at least one of the plurality of struts of the distal framework section or the proximal framework section.

The at least one start protrusion section is preferably attached to the coupling element.

The medical occlusion device preferably has a pulling element for drawing the medical occlusion device into a lumen of a catheter.

A catheter system includes the medical occlusion device of any of the previous examples and a catheter.

A medical occlusion device for occluding a body lumen or a cardiovascular structure, for example for occluding a left atrial appendage, includes
a framework having a longitudinal framework axis wherein the framework has a compressed framework state and is configured to expand in an expanded framework state when the medical occlusion device is not being compressed, and wherein the framework has a distal framework section, a proximal framework section and an intermediate framework section between the proximal framework section and the distal framework section,
a cover covering at least an outer side of the proximal framework section, and
at least one protrusion for positioning and/or anchoring the medical occlusion device at an ostium of the body lumen or the cardiovascular structure, for example at an ostium of the left atrial appendage, having a start protrusion section, a middle protrusion section and an end protrusion section, and wherein at least the end protrusion section projects radially beyond the framework in its expanded framework state, and wherein
a) at least the start protrusion section is attached to or protrudes from the proximal framework section and at least the end protrusion section protrudes through the cover, or
b) at least the end protrusion section is adjacent to the proximal framework section in its expanded framework state with respect to the longitudinal framework axis and at least the start protrusion section is attached to or protrudes from the intermediate framework section and/or the distal framework section.

The end protrusion section is preferably not covered by the cover.

The framework preferably has a plurality of struts.

The at least one protrusion is preferably at least partially covered by the cover and at least one section of the protrusion is directly attached to the proximal framework section.

The start protrusion section is preferably partially covered by the cover and the start protrusion section is directly attached to the outer side of the struts of the proximal framework section.

The end protrusion section and/or the middle protrusion section or at least a part of the middle protrusion section preferably protrudes through the cover.

The start protrusion section is preferably directly attached to the inner side of the plurality of struts of the proximal framework section.

The at least one protrusion is preferably not covered by the cover and the start protrusion section is directly attached to the inner side of the proximal framework section.

The plurality of struts preferably forms a cells structure having a plurality of cells or a net structure having a plurality of meshes.

The cells of the cell structure are preferably diamond-shaped cells.

The plurality of struts can be in the form of a wire or strip.

The plurality of struts preferably extends from the proximal framework section to the distal framework section or vice versa.

The plurality of struts preferably extends along a longitudinal framework axis in the compressed framework state.

The plurality of struts is preferably at least partially made of a shape-memory material.

The plurality of struts preferably consists of or include a Nickel-Titanium alloy, a Cobalt-Chromium alloy, a polymer or a stainless steel.

The framework or the plurality of struts are preferably produced by laser cutting from a shape-memory material.

The framework preferably has a closed framework structure wherein the distal framework section has a crossing point with the longitudinal framework axis or wherein the framework has an open framework structure wherein the distal framework section has no crossing point with the longitudinal framework axis.

The framework preferably encases an inner framework volume in the form of a sphere, a flattened sphere, preferably an ellipsoid.

The framework is preferably a self-expandable or self-expanding framework.

The medical occlusion device preferably has at least 1, 2 or 3 protrusions, preferably 2 to 24 protrusion, more preferably 3 protrusions.

A protrusion has at least one, preferably 1 or 2, end protrusion sections and at least one, preferably 1 or 2, middle protrusion sections and at least one, preferably 1 or 2, start protrusion sections.

At least one middle protrusion section preferably has an inflection point with respect to the start protrusion section and the end protrusion section.

The end protrusion section is preferably angled by an angle between 70° and 110°, preferably between 80° and 100°, more preferably between 85° and 95°, most preferably by an angle of 90, with respect to the middle protrusion section and/or the start protrusion section in the expanded framework state.

The at least one protrusion is preferably a distinct protrusion.

The at least one protrusion is preferably not directly attached to each other.

The at least one protrusion may include or consist of a radiopaque material or at least one radio marker is attached thereto.

The at least one protrusion is not a circumferential protrusion with respect to the longitudinal framework axis.

A dimension of the protrusion in longitudinal direction is preferably greater than a dimension of the framework in longitudinal direction in the expanded framework state and/or in the compressed framework state.

The at least one end protrusion section is preferably blunt.

The at least one end protrusion section preferably has rounded corner(s) and/or edge(s) and/or tip(s).

The at least one protrusion can be a wire, strap or a strip.

The at least one protrusion includes a winding.

The at least one protrusion is preferably a loop, preferably a closed or open loop.

The at least one protrusion can have an airfoil-likes shape, or wing-like shape or lasso-like shape in its expanded state or looks like the cross section through a mushroom or an umbrella.

The at least one end protrusion section is preferably u-shaped, n-shaped, c-shaped, elliptical shaped or circular shaped.

The end protrusion sections are preferably evenly distributed in circumferential direction.

The start protrusion section of one protrusion or several protrusions can be attached to different framework sections or to the same framework section at different heights with respect to the longitudinal framework axis.

The middle protrusion section of the at least protrusion is preferably at the same level as the proximal framework section with respect to the longitudinal framework axis.

The at least one is preferably self-expendable or self-expanding.

At least one protrusion preferably extends along the dimension of the framework and back in the longitudinal direction in the expanded state.

At least one start protrusion section is preferably attached to the inner side of the framework or the inner side of the struts.

At least one start protrusion section is preferably directly attached to the outer side of the struts.

At least one protrusion preferably has a section extending within the inner framework volume and another section extending outside the framework in the expanded state.

The shape in cross section of the at least one protrusion is preferably circular, elliptic or polygonal with rounded corners.

The at least one start protrusion section is preferably partially covered by the cover.

The at least one protrusion preferably consists of or includes a shape-memory material.

The at least one protrusion preferably consists of or includes a Nickel-Titanium alloy, a Cobalt-Chromium alloy, a polymer or a stainless steel.

The at least one protrusion and the framework or at least one of the struts of the framework are preferably fixed together by welding, screwing or riveting both together.

The cover is preferably made of flexible material.

The cover preferably includes or consists of a material impermeable for blood clots or thrombi but permeable for blood.

The cover preferably includes or consists of a polymer or a biological tissue, preferably a human and/or animal tissue.

The cover preferably includes or consists of a pericardial tissue, preferably decellularized pericardial tissue.

The end protrusion section is preferably not covered by the cover.

Each of the at least one protrusion preferably two times penetrates the cover.

The cover preferably covers at least one third of the framework at its proximal framework section or the full framework in the expanded state.

The cover preferably covers the outer side of the struts and spaces between the struts in the expanded framework state.

The cover preferably includes at least one or springs being sized and configured to close-off a through-hole of the cover where the at least one protrusion or the coupling element penetrates the cover.

The cover is preferably glued or sutured to the plurality of struts.

The plurality of struts is preferably embedded in the cover.

The medical occlusion device preferably includes a coupling element for coupling to a catheter.

The coupling element is preferably attached to the distal framework section or the proximal framework section.

The coupling element is preferably attached to at least one of the plurality of struts of the distal framework section or the proximal framework section.

The at least one start protrusion section is preferably attached to the coupling element.

The medical occlusion device preferably has a pulling element for drawing the medical occlusion device into a lumen of a catheter.

The cover preferably includes at least one spring being sized and configured to close-off a through-hole of the cover where the at least one protrusion protrudes through the cover.

A system includes the medical occlusion device of any of the previous examples and a catheter with an inner tube configured to receive the medical occlusion device in the compressed framework state.

The medical occlusion device is preferably detachably connected to the catheter.

The medical occlusion device and the inner tube of the catheter are preferably detachably connected by screwing, releasing anchors, a bayonet lock or via a predetermined breaking point.

The medical occlusion device can be used in closing a body lumen or a cavity, preferably a left atrial appendage.

A system of the invention can be used in closing a body lumen or a cavity, preferably a left atrial appendage, is described.

A medical occlusion device for occluding (an abnormality or defect in) a body lumen or a cardiovascular structure, for example for occluding a left atrial appendage, includes
a framework having a longitudinal framework axis wherein the framework has a compressed framework state and is configured to expand in an expanded framework state when the medical occlusion device is not being compressed, and wherein the framework has a distal framework section, a proximal framework section and an intermediate framework section between the proximal framework section and the distal framework section,
a cover covering at least an outer side of the proximal framework section (at least in the expanded framework state), and
at least one protrusion for positioning and/or anchoring the medical occlusion device at an ostium of the body lumen or the cardiovascular structure, for example at an ostium of the left atrial appendage, having a start protrusion section, a middle protrusion section and an end protrusion section, and wherein at least the end protrusion section projects radially beyond the framework in its expanded framework state (with respect to the longitudinal axis), and wherein at least the start protrusion section is directly attached to or protrudes from the proximal framework section and at least the end protrusion section protrudes through the cover.

A medical occlusion device for occluding (an abnormality or defect in) a body lumen or a cardiovascular structure, for example for occluding a left atrial appendage, includes
a framework having a longitudinal framework axis wherein the framework has a compressed framework state and is configured to expand in an expanded framework state when the medical occlusion device is not being compressed, and wherein the framework has a distal framework section, a proximal framework section and an intermediate framework section between the proximal framework section and the distal framework section,
a cover covering at least an outer side of the proximal framework section (at least in the expanded framework state), and
at least one protrusion for positioning and/or anchoring the medical occlusion device at an ostium of the body lumen or the cardiovascular structure, for example at an ostium of the left atrial appendage, having a start protrusion section, a middle protrusion section and an end protrusion section, and wherein at least the end protrusion section projects radially beyond the framework in its expanded framework state with respect to the longitudinal axis, and wherein at least the end protrusion section is adjacent to the proximal framework section in its expanded framework state with respect to the longitudinal framework axis, and at least the start protrusion section is directly attached to or protrudes from the intermediate framework section and/or the distal framework section.

A medical occlusion device for occluding (an abnormality or defect in) a body lumen or a cardiovascular structure, for example for occluding a left atrial appendage, includes:
a first device part includes
   a framework having a longitudinal framework axis wherein the framework has a compressed framework state and is configured to expand in an expanded framework state when the medical occlusion device is not being compressed, and wherein the framework has a distal framework section, a proximal framework section and an intermediate framework section between the proximal framework section and the distal framework section,
   a cover covering at least the proximal framework section (at least in the expanded framework state), and
   a first connector part, and second device part including:
   a second connector part, being configured to be connectable with the first connector part of the first device part,
   at least one protrusion for positioning and/or anchoring the medical occlusion device at an ostium of the body lumen or the cardiovascular structure, for example at an ostium of the left atrial appendage, having a start protrusion section, a middle protrusion section and an end protrusion section, and wherein at least the end protrusion section projects radially beyond the framework in its expanded framework state with respect to the longitudinal axis, and wherein the start protrusion section of the at least one protrusion is attached to the second connector part.

The first connector part may be located along the longitudinal axis, preferably in the distal framework section.

A further aspect relates to a system including any of the medical occlusion devices as described in this application and a catheter with an inner tube configured to receive the medical occlusion device in the compressed framework state.

The medical occlusion device may be detachably connected to the catheter.

The medical occlusion device and the inner tube of the catheter may be detachably connected by screwing, releasing anchors, a bayonet lock or via a predetermined breaking point.

A further aspect relates to a medical occlusion device or a system as described for use of a closure of a body lumen or a cavity, preferably a left atrial appendage, is described.

Also described is a method of closure of a left atrial appendage a medical occlusion using the medical occlusion device of example 140, 141 or 142 or any other medical occlusion device described in this application.

A method for implanting the medical occlusion device of examples 140, 141 or 142 or any other medical occlusion device described in this application into a human or animal body is described as well. Such a method is preferably a method for left atrial appendage occlusion (LAAO) or left atrial appendage closure (LAAC).

Also described is a method for implanting the medical occlusion device and optionally any other features described in this application, wherein the second device part is positioned and/or anchored at an ostium of a body lumen or a cardiovascular structure, for example at an ostium of a left atrial appendage, in a first implantation step and in a subsequent second implantation step connecting the first connector of the first device part to the second connector part of the second device part. The method may further include a subsequent third implantation step wherein the first device part is moved out of the catheter and released from the catheter, thereby expanding the framework of first device part into the expanded framework state. The first device part may be housed in a first catheter and the second device part is housed in a second catheter or the first device part and the second device part are housed in the same catheter.

In the following figures the invention is explained with regard to the closure of a LAA. However, other body lumina, cavities or cardiovascular structures may be occluded in the same manner.

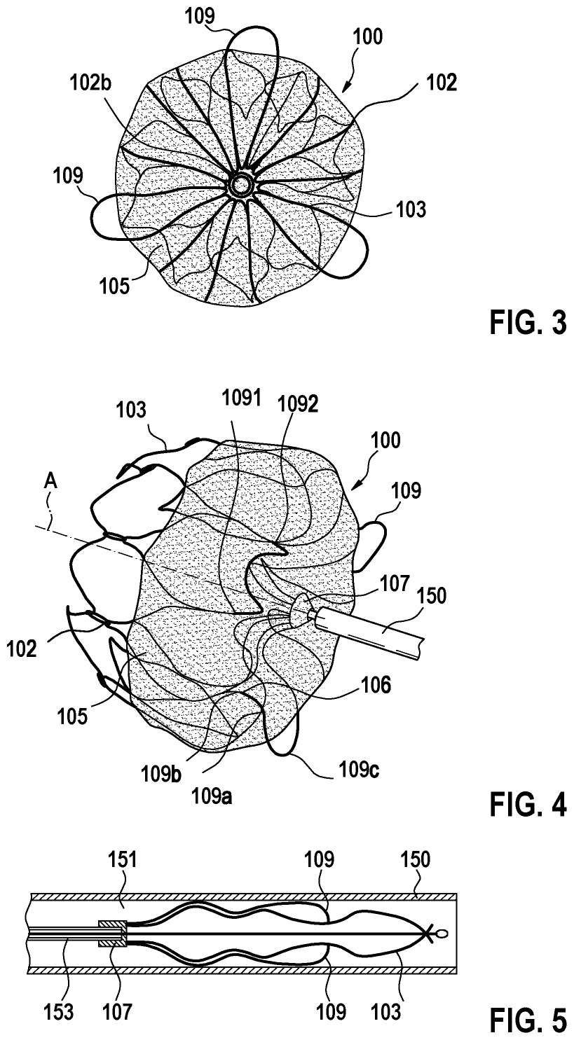

FIGS. 2 to 4 show a first embodiment of a medical occlusion device 100 for occluding an LAA 3 of a heart, in particular a human heart, in an expanded state, wherein this medical occlusion device 100 is depicted in FIG. 5 in a compressed state. The LAA 3 includes an ostium 4 and an inner surface 5 (of the ostium 4/ the LAA 3). A rim 7 is formed at the ostium 4 where the inner surface 5 of the LAA 3 merges with an inner surface 9 of a left atrium 10.

The medical occlusion device 100 has a self-expandable stent framework 102 with a distal framework section 102a and a proximal framework section 102b including a plurality of (Nitinol) struts 103, wherein the framework 102 expands automatically from a compressed state into an expanded state due to the shape memory effect when being unconstrained. The struts 103 form a network of cells and encase a (flattened spherically shaped) volume in the expanded state (see FIGS. 2 to 4). Further, the medical occlusion device 100 includes a (flexible) cover 105 fixed to the framework 102, preferably at its outer side. The cover 105 as shown is in unfolded or stretched in the expanded state of the framework 102. The cover 105 covers (approximately) half of the outer side of the framework 102 but at the proximal framework section. The cover 105 cover the proximal framework section and at least partially the intermediate framework section. The cover may be made of a polymer material or a biological tissue, e.g. (decellularized) pericardial tissue.

During implantation the medical occlusion device 100 is attached to a catheter 150 with a lumen 151 for receiving the medical occlusion device 100 in the compressed state of the framework 102, wherein the medical occlusion device 100 may be released by being pushed out from the lumen 151 (in the distal direction or to the right in FIG. 5) so that the framework 102 is transferred into the expanded state, which is shown in FIGS. 2 to 4. The medical occlusion device 100 is attached to the catheter 150 via a coupling element 107. The medical occlusion device 100 may have a flexible and in particular limp and elongate pulling element (not shown) fixed to the framework, such that the medical occlusion device 100 may be pulled back again into the lumen 151 of the catheter 150 by means of the pulling element, and in so doing the framework 102 may be transferred into the compressed state (see FIG. 5).

In order to push out the medical occlusion device 100, the slider 153 (also known as a pusher) is used, which may be arranged in the lumen 151 of the catheter 150 and is movable in the lumen 151 (shown in FIG. 5). For example, the slider 153 may include a polymer tube, a hypotube, or what is known as a push-wire. After correct arrangement of the medical occlusion device 100 the medical occlusion device is decoupled from the catheter 150 at coupling 107, for example by disconnection of a bayonet connection.

The medical occlusion device 100 further includes three loops 109 attached with its first part and its second part to adjacent struts 103 of the framework 102. The (Nitinol) loops 109 may be, for example, welded to the struts 103. Each loop 109 penetrates the cover 105 two times at respective through holes 106 (see FIG. 4). Each loop 109 has two start loop sections 109b, namely a first start loop section 1091 and a second start loop section 1092, running generally parallel to a longitudinal framework axis A (see FIG. 2) of the framework 102 or slightly inclined thereto and two middle loop sections 109a which are bent with regard to the adjacent start loop sections 109b by approximately 110°. An end loop section 109c, preferably having an U-shape, is connected to the two middle loop sections 109a and each middle loop section 109a is connected to one of the two start loop sections 109b. As one can derive from FIGS. 2 to 4 the end loop section 109c of the loop 109 extends beyond the framework 102 in longitudinal direction and in radial direction with regard to the longitudinal framework axis A. This shape allows the medical occlusion device 100 to be fixed at the ostium 4 of the LAA 3. In particular, the distal loop section and the middle loop section 109a abuts the rim 7 of the LAA 3. Thereby, a correction position of the medical occlusion device 100 is easily achieved, wherein the loops 109 hold the medical occlusion device in place and prevent it from pulling too deep into the LAA 3. The sliding of the medical occlusion device 100 too deep into the LAA 3 is blocked by the ostium 4 and the end loop sections 109c of the loops 109 anchoring at the rim 7 of the atrium 10. The framework 102 with the cover 105 occludes the LAA 3 so that thrombi formed in the LAA 3 are prevented from moving into the atrium 10. For that the struts 103 located more proximally and the cover 105 abut the inner surface 5 of the LAA 3 and are biased into the direction perpendicular to the inner surface 5.

In FIG. 5 the medical occlusion device 100 is arranged within a lumen 151 of a catheter 150 in a compressed state of the framework. A slider 153 (or pusher) may be coupled to a coupling element 107 of the medical occlusion device. The loops 109 fixed to struts 103 may be shorter than the whole framework in the compressed state. The cover 105 is not shown in FIG. 5 for clarity reasons. Before releasing the medical occlusion device from the catheter lumen, the struts may fixed at a delivery system

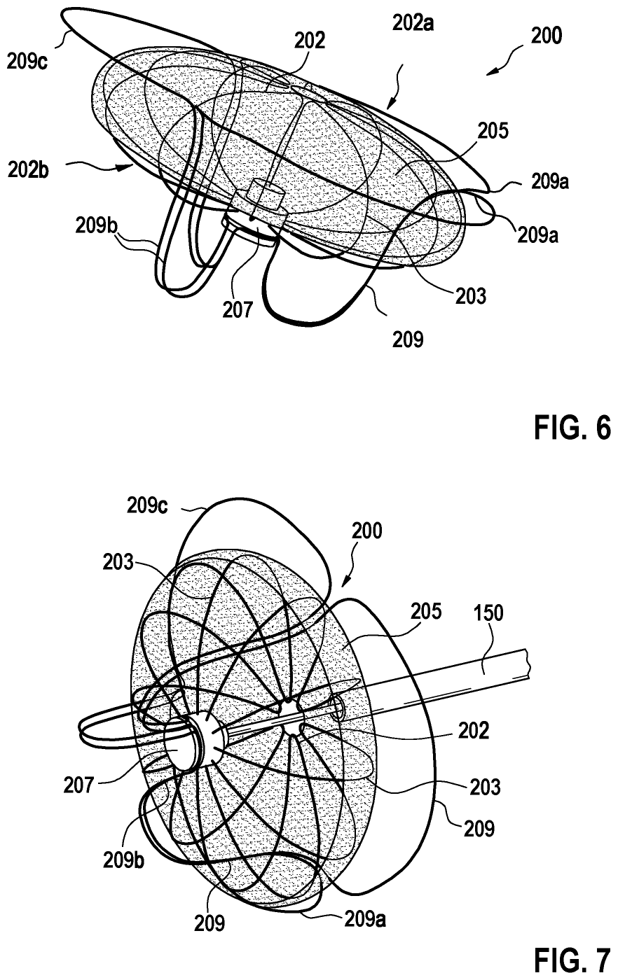

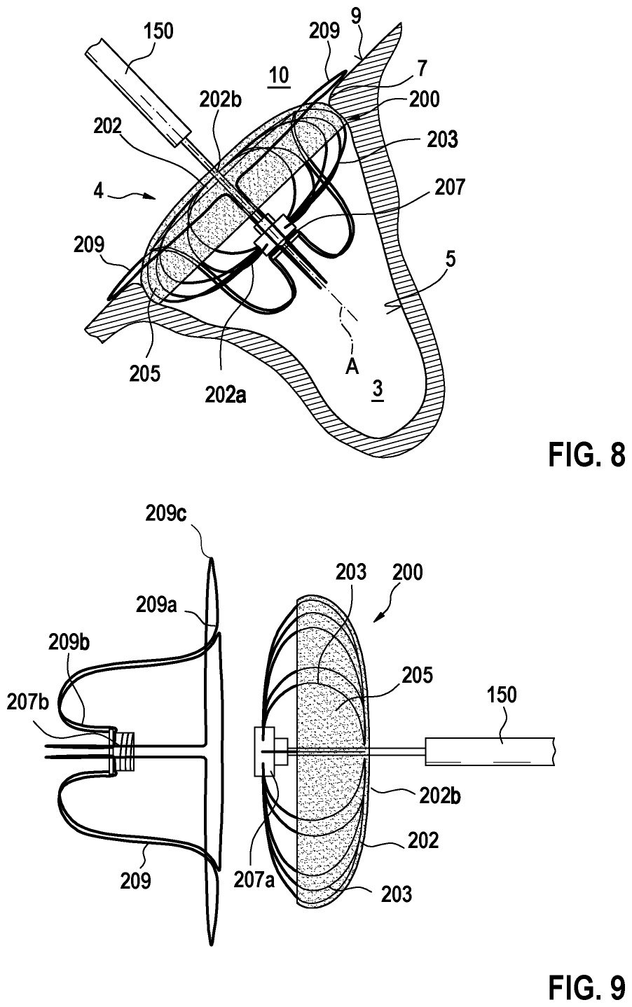

A second embodiment of a medical occlusion device 200 shown in FIGS. 6 to 9 has a self-expandable stent framework 202 including a plurality of (Nitinol) struts 203, expanding automatically from a compressed state into an expanded state due to the shape memory effect. The framework has a distal framework section 202a and a proximal framework section 202b. The struts 203 have a spoke-like or rib-like form and encase a flattened spherically-formed volume in the expanded state (see FIGS. 6 to 9). Further, the medical occlusion device 200 includes a (flexible) cover 205 fixed to the framework 202 at its outer side, wherein the cover 205 is unfolded or stretched in the expanded state of the framework 202. The cover 205 covers approximately half of the outer side of the framework 202 at the proximal side.

The medical occlusion device 200 further includes three protrusions (e.g. loops) 209 with an end protrusion (e.g. loop) section 209c, two middle protrusion (e.g. loop) 209a, and two start protrusion (e.g. loop) sections 209b. The start protrusion (e.g. loop) section 209b is attached to the distal framework section 202a of the framework 202 at a connector 207 for the catheter 150. The protrusions (e.g. loops) 209 may be, for example, welded to the connector 207. The protrusions (e.g. loops) 209 may be made of a nickel titanium alloy like Nitinol. In particular, as depicted in FIG. 9, the connector 207 may be composed of two connector parts 207a, 207b. A first connector part 207a is located at the distal framework section 202a of the framework 202 and the second connector part 207b is fixed to the first part and the second part of each loop. During treatment the medical occlusion device may be implanted in two steps. In the first step the protrusions (e.g. loops) 209 with the second part 207b of the coupling element 207 may be implanted and correctly placed at the ostium 4 of the LAA 3 as indicated below. In the second step the framework 202 with the first connector part 207a of the connector 207 may be implanted and fixedly attached to the second connector part 207b of the connector 207, for example by screwing.

Each protrusion (e.g. loop) 209 has two start protrusion (e.g. loop) sections 209b running firstly parallel to a longitudinal framework axis A (see FIG. 6) of the framework 202 or slightly inclined (<20°) thereto then having a turning point and then running again parallel to a longitudinal framework axis A of the framework 202 or slightly inclined (<20°) thereto. Each loop has a two middle protrusion (e.g. loop) sections 209a which are bent with regard to the adjacent start protrusion (e.g. loop) sections 209b by approximately 90°. Accordingly, the protrusion (e.g. loop) 209 has a S-shape formed by the middle protrusion (e.g. loop) section 209a, the start protrusion (e.g. loop) section 209b and the end protrusion (e.g. loop) section 209c. As one can derive from FIGS. 6 to 8 the middle protrusion (e.g. loop) section 209a of the protrusion (e.g. loop) 209 extends beyond the framework 202 in radial direction with regard to the longitudinal framework axis A. Further, the protrusion (e.g. loop) 209 is approximately at the same level as the proximal framework section 202 with regard to the longitudinal framework axis A. This shape allows the medical occlusion device 200 to be fixed at the ostium 4 of the LAA 3. In particular, the middle protrusion (e.g. loop) section 209a abuts at the inner surface 9 of the atrium 10. Thereby, a correct position of the medical occlusion device 200 is easily achieved, wherein the protrusions (e.g. loops) 209 hold the medical occlusion device 200 in place and prevent it from pulling too deep into the LAA 3. The sliding of the medical occlusion device 200 too deep into the LAA 3 is blocked by the middle protrusion (e.g. loop) section 209a anchoring at the inner surface 9 of the atrium 10. The framework 202 with the cover 205 occludes the LAA 3 so that thrombi formed in the LAA 3 are prevented from moving into the atrium 10. The through-hole within the cover 205 for penetration of the catheter 150 is closed at the end of the implantation procedure by springs or any other suitable means.

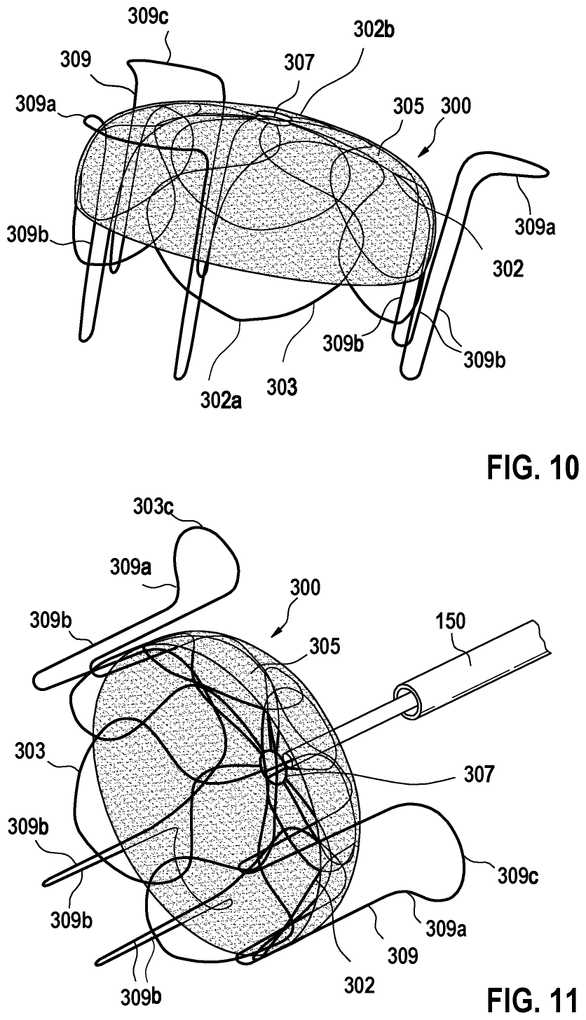

A third embodiment of a medical occlusion device 300 shown in FIGS. 10 to 14 has a self-expandable stent framework 302 with a distal section 302a and a proximal section 302b including a plurality of (Nitinol) struts 303, expanding automatically from a compressed state into an expanded state due to the shape memory effect. The struts 303 form a net-like structure with a plurality of cells and encase a cylindrically formed volume in the expanded state (see FIGS. 10 to 13). Further, the medical occlusion device 300 includes a (flexible) cover 305 fixed to the framework 302 at its outer surface, wherein the cover 305 is unfolded or stretched in the expanded state of the framework 302. The cover 305 can include a polymer material or a biological tissue (e.g. pericardial tissue) covering approximately half of the outer surface of the framework 302 at the proximal side.

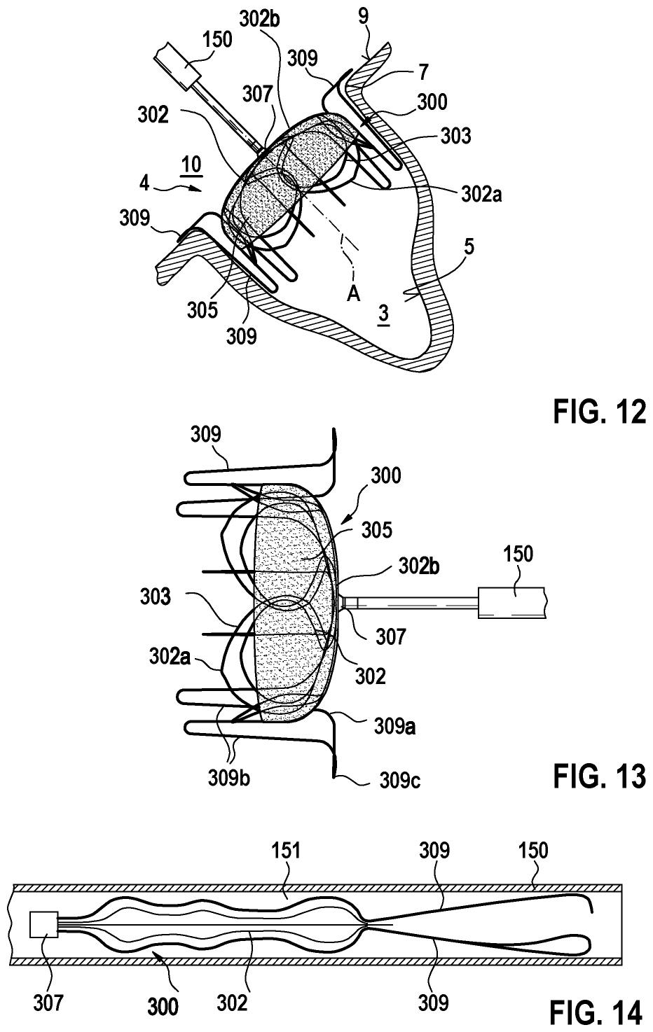

The medical occlusion device 300 further includes two loops 309 (see FIGS. 12 and 13) or three loops 309 (see FIGS. 10 and 11) attached with its start loop section 302b to the framework 302 and/or a coupling 307 for the catheter 150. The (Nitinol) loops 309 may be, for example, welded to the coupling element 307. The implantation procedure may be similar to the one of the medical occlusion device of the first embodiment.

Each loop 309 has two start loop sections 309b running generally parallel to a longitudinal framework axis A (see FIG. 10) of the framework 302 or slightly inclined thereto having a turning point and then running again parallel to a longitudinal framework axis A of the framework 302 or slightly inclined (<20°) thereto. Each loop has further two middle loop sections 309a which are bent with regard to the adjacent start loop sections 309b by approximately 90° and one end loop section 309c being adjacent to the two middle loop section 309a. Accordingly, the distal loop section 309c has an U-shape formed. As one can derive from FIGS. 10, 12 and 13 the middle section 309a of the loop 309 extends beyond the framework 302 in radial direction and in longitudinal direction with regard to the longitudinal framework axis A. This shape allows the medical occlusion device 300 to be fixed at the ostium 4 of the LAA 3. In particular, the middle section 309a abuts at the inner surface 9 of the atrium 10. Thereby, a correct position of the medical occlusion device 300 is easily achieved, wherein the loops 309 hold the medical occlusion device in place and prevent it from pulling too deep into the LAA 3. The sliding of the medical occlusion device 300 too deep into the LAA 3 is blocked by the middle section 309a of the loop 309 anchoring at the inner surface 9 of the atrium 10. The framework 302 with the cover 305 occludes the LAA 3 so that thrombi formed in the LAA 3 are prevented from moving into the atrium 10. The distance between the protrusion 309 and the cover 305 and to the inner surface 5 of the LAA 3 shown in FIG. 12 is for clarity reasons only (to show the form of the loops 309). Naturally, the framework 302 with the cover 302 and the protrusions 309 abuts the inner surface 5 of the LAA 3 in the final position in order to occlude the LAA 3. The distance between the protrusion 309 and the cover 305 shown in FIG. 13 note necessarily there and could be also only for clarity reasons. However, in the not implanted state there might be a distance between the framework and the protrusion, as long as this distance is eliminated in the implanted state due to the pressure applied from the LAA walls to the protrusion and the framework.

FIG. 14 shows a catheter system including the medical occlusion device 300 and a catheter 150. As one can derive from FIG. 14 showing the compressed state of a medical occlusion device 300 the loops 309 are longer than whole the framework 302 in the compressed state. The medical occlusion device 300 is situated in the lumen 151 of the catheter 150 for receiving the medical occlusion device in the compressed state of the framework 302. The medical occlusion device 300 is attached to the catheter 150 via a coupling element 307. The cover 305 is not shown in FIG. 14 for clarity reasons. The medical occlusion device 300 may have a pulling element (not shown) fixed to the framework via the coupling element 307, such that the medical occlusion device 300 may be pulled back into the lumen 151 of the catheter 150 by means of the pulling element, and in so doing the framework 302 may be transferred into the compressed state. The medical occlusion device 100 can be released from the catheter by being pushed out from the lumen 151 (in the distal direction or to the right in FIG. 5) so that the framework 302 is transferred into the expanded state. Accordingly, during implantation the loops 309 are released first if the outer sleeve is retracted and the medical occlusion device 300 may be correctly positioned at the ostium first (see FIG. 12) before the whole framework 302 is expanded.

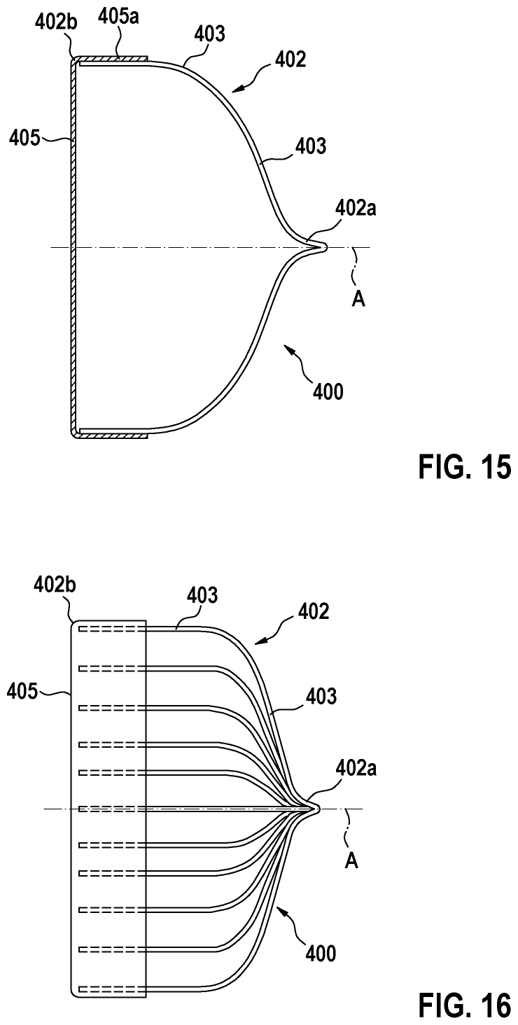

FIGS. 15 and 16 show another embodiment of the medical occlusion device 400 to which loops of the first embodiment or the second embodiment may be used for correct positioning at the LAA ostium. The loops 109 or 209 are formed similarly and attached analogously as explained above with respect to the first embodiment and the second embodiment of the medical occlusion device 100, 200.

As can be seen in FIGS. 15 and 16, the stent framework 402 has a plurality of struts 403, wherein the framework 402 has a distal section 402a and an opposite proximal section 402b, wherein at the distal section 402a the struts 402 are connected to one another, and wherein at the proximal section 402b the struts 403 are connected to a peripheral edge region 405a of the cover 405. Here, the struts 403 run-based on an expanded state of the stent framework 402—starting from the distal section 402a of the framework 402 in an axial direction A and in each case along a certain section in a radial direction perpendicular thereto, so that the struts 403 run away from one another in the direction of the proximal section 402b of the framework 402. In this expanded state the cover 405 fixed to the struts 403 is unfolded or stretched. In contrast, in the compressed state of the framework 402 the struts 403 have a much shorter spacing from one another in the radial direction, so that the material layer 405 is not stretched.

In order to fix the peripheral edge region 405a of the cover 405 to the struts 403, the respective end portions of the struts may have at least one recess (not shown), for example an eyelet, wherein the peripheral edge region 405a of the cover 405 is connected to the at least one recess, for example by means of one seam or one thread per recess/strut.

The above examples of medical occlusion devices 100, 200, 300, 400 with loops 109, 209, 309 and of a catheter 150 significantly facilitate the alignment and apposition of the respective medical occlusion device at the LAA ostium. Accordingly, the time requirement for implantation is lowered and herewith implantation costs. Further, reliable fixation of the medical occlusion device in the ostium is provided while avoiding sliding of the medical occlusion device into the LAA.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A medical occlusion device for occluding a body lumen or a cardiovascular structure, comprising:
a framework comprising a longitudinal framework axis, (A), wherein the framework comprises a compressed framework state and is configured to expand in an expanded framework state when the medical occlusion device is not being compressed, and wherein the framework comprises a distal framework section, a proximal framework section and an intermediate framework section between the proximal framework section and the distal framework section,
a cover covering at least the proximal framework section,
a first connector part connected to the framework, wherein the first connector part is located in the distal framework section,
a second connector part configured to be connectable with the first connector part,
at least one protrusion configured to position and/or anchor the medical occlusion device at an ostium of the body lumen or the cardiovascular structure, the protrusion comprising at least one start protrusion section, at least one middle protrusion section and at least one end protrusion section, wherein at least one of the at least one end protrusion sections projects radially beyond the framework in its expanded framework state with respect to the longitudinal framework axis, wherein the start protrusion section is attached to the second connector part, and wherein the proximal framework section comprises a coupling element configured to provide a releasable connection with a catheter.

2. The medical occlusion device of claim 1, wherein the first connector part and/or the second connector part is located along the longitudinal framework axis.

3. (canceled)

4. The medical occlusion device of claim 1, wherein the framework comprises a plurality of struts, extending from the proximal framework section to the distal framework section or vice versa and/or wherein the framework is a self-expandable or self-expanding framework.

5. (canceled)

6. The medical occlusion device of claim 1, wherein the end protrusion section is angled by an angle between 70° and 110° with regard to the middle protrusion section and/or the start protrusion section in the expanded framework state.

7. The medical occlusion device of claim 1, wherein a dimension of the at least one protrusion in a longitudinal direction is greater than the dimension of the framework in the longitudinal direction in the expanded framework state and/or in the compressed framework state.

8-9. (canceled)

10. The medical occlusion device of claim 1, wherein the at least one protrusion is an open or closed loop.

11. The medical occlusion device of claim 1, wherein the framework is a self-expandable or self-expanding framework.

12. The medical occlusion device of claim 4, wherein the coupling element is attached to at least one of the plurality of struts.

13. The medical occlusion device of any of claim 1, wherein the coupling element is situated at the proximal framework section.

14-21. (canceled)

22. The medical occlusion device of claim 1, wherein the framework comprises a closed framework structure wherein the distal framework section has a crossing point with the longitudinal framework axis or wherein the framework comprises an open framework structure wherein the distal framework section has no crossing point with the longitudinal framework axis and/or wherein the framework (202) encases an inner framework volume in the form of a sphere or a flattened sphere.

23-24. (canceled)

25. The medical occlusion device of claim 1, wherein the framework has not a tubular structure or stent form.

26. (canceled)

27. The medical occlusion device of claim 1, wherein the middle protrusion comprises an inflection point with respect to the start protrusion section and the end protrusion section.

28-29. (canceled)

30. The medical occlusion device of claim 1, wherein the at least one protrusion comprises or consists of a radiopaque material or at least one radio marker is attached to the at least one protrusion.

31. The medical occlusion device of any claim 1, wherein the at least one protrusion comprises a non-circumferential protrusion with respect to the longitudinal framework axis.

32. The medical occlusion device of claim 1, wherein one or more of the end protrusion section, the middle protrusion section and the start protrusion section is blunt and/or wherein the end protrusion section is u-shaped, n-shaped, c-shaped, elliptical shaped or circular shaped.

33-40. (canceled)

41. The medical occlusion device of claim 1, wherein the at least one protrusion is self-expendable or self-expanding and/or wherein the at least one protrusion consists of or comprises a shape-memory material and/or consists of or comprises a Nickel-Titanium alloy, a Cobalt-Chromium alloy, a polymer or a stainless steel.

42-50. (canceled)

51. The medical occlusion device of any of claim 1, wherein the cover is made of flexible material and/or the cover comprises or consists of a material impermeable for blood clots or thrombi but permeable for blood and/or the cover comprises or consists of a polymer or a biological tissue, a human/or animal tissue and/or wherein the cover comprises or consists of a pericardial or a decellularized pericardial tissue.

52-56. (canceled)

57. The medical occlusion device of claim 1, wherein the cover covers at least one third of the framework at its proximal framework section or the full framework in the expanded state and/or wherein the cover covers an outer side of the struts and spaces between the struts in the expanded framework state and/or wherein the cover is glued or sutured to the plurality of struts, or wherein the plurality of struts are embedded in the cover.

58-63. (canceled)

64. The medical occlusion device of claim 1, wherein the coupling element is attached to the distal framework section or the proximal framework section, and/or wherein the coupling element is attached to at least one of a plurality of struts of the distal framework section or the proximal framework section, and or wherein the at least one start protrusion section is attached to the coupling element.

65-67. (canceled)

68. A catheter system comprising the medical occlusion device of claim 1 and a catheter.

69-139. (canceled)
